(19) United States
(12) Patent Application Publication
(10) Pub. No.: US 2014/0199401 A1
(43) Pub. Date: Jul. 17, 2014

(54) EXTENDED RELEASE PHARMACEUTICAL COMPOSITIONS OF FESOTERODINE

(71) Applicants: Sunilendu Bhushan Roy, Ahmedabad (IN); Sushrut Krishnaji Kulkarni, Ahmedabad (IN); Ajay Kumar Handa, Ahmedabad (IN); Ketan Tulsidas Savjani, Ahmedabad (IN)

(72) Inventors: Sunilendu Bhushan Roy, Ahmedabad (IN); Sushrut Krishnaji Kulkarni, Ahmedabad (IN); Ajay Kumar Handa, Ahmedabad (IN); Ketan Tulsidas Savjani, Ahmedabad (IN)

(73) Assignee: CADILA HEALTHCARE LIMITED, Ahmedabad (IN)

(21) Appl. No.: 14/130,556

(22) PCT Filed: Jul. 4, 2012
(86) PCT No.: PCT/IN2012/000470

§ 371 Date: Jan. 2, 2014

(30) Foreign Application Priority Data
Jul. 4, 2011 (IN) 1927/MUM/2011

(51) Int. Cl. A61K 47/36; A61K 47/38; A61K 47/26; A61K 31/222; A61K 9/14

(52) U.S. Cl. CPC A61K 47/36; A61K 31/222; A61K 9/14; A61K 47/26; A61K 47/38
USPC 424/490; 514/546; 424/489

(57) ABSTRACT

A stable extend release pharmaceutical composition is disclosed. The composition comprises particles of fesoterodine or salts thereof, one or more rate controlling polymers and one or more pharmaceutically acceptable excipients wherein at least 90% of the total amount of particles of fesoterodine or salts thereof by volume (D₉₀) are having size greater than about 200 microns

### FIELD OF THE INVENTION

The present invention relates to stable extended release pharmaceutical compositions of fesoterodine or salts thereof. The invention also relates to processes for the preparation of such compositions and use thereof for treatment of overactive bladder or patient having symptoms of urinary incontinence, urinary urge incontinence, imperative urinary urge, and/or increased urinary frequency.

### BACKGROUND OF THE INVENTION

Fesoterodine can be chemically described as 2-[(1R)-3-(diisopropylamino)-1-phenylpropyl]-4-(hydroxymethyl)phenyl isobutyrate, and having the following structural formula:

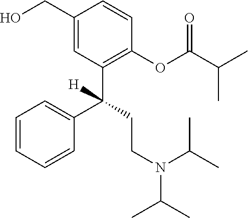

Fesoterodine is an innovative drug for the treatment of overactive bladder, urinary incontinence and other dysfunctions of the urinary tract. It is disclosed, inter alia, in European Patent No. EP 1077912 B1, pertaining to novel derivatives of 3,3-diphenylpropylamines. European Patent No. EP 1230209 B1 discloses stable salts of novel derivatives of 3,3-diphenylpropylamines, including Fesoterodine hydrogen fumarate.

Fesoterodine is currently marketed in United States as TOVIAZ® tablets by Pfizer. Each TOVIAZ® tablet contains 4 or 8 mg of fesoterodine fumarate.

Overactive bladder (OAB) is an extremely common disorder, affecting 17% of the adult population in major European countries. OAB can occur at any age and in either gender, although its prevalence is higher in geriatric and female populations.

OAB is a bladder function disorder resulting in symptoms of urgency, with or without urge incontinence, and usually includes increased urinary frequency and nocturia. The disorder is due to spastic contractions of the detrusor muscle of the bladder, resulting in sustained high bladder pressure and the urgent need to urinate.

This can be caused by several reasons, such as traumatic or toxic nerve damage (e.g., abdominal trauma, pelvic trauma or surgery, bladder stones, adverse effects of drugs), neurological diseases (e.g., spinal cord lesions, multiple sclerosis, Parkinson's disease, excessive neurotransmitter release in the bladder) or myogenic instability (e.g., bladder hypertrophy caused by outlet obstruction or urinary tract infection).

In some cases, OAB can be managed without pharmacotherapy, using exercise, pessaries, implants, biofeedback or behavioral therapy. But in most cases, pharmacotherapy is the better option. Antimuscarinic agents have been found to be particularly effective for treating OAB. During normal micturition, acetylcholine released from postganglionic parasympathetic neurons acts on the muscarinic receptors of the detrusor smooth muscle in the bladder to stimulate contractions. Antimuscarinic agents interfere with this action, thus reducing detrusor contractions. However, despite the availability of different antimuscarinic drugs, physicians and patients remain dissatisfied with current treatments due to adverse events and/or insufficient efficacy. Furthermore, as a general matter, it is desirable for pharmaceutical compounds to have as little effect on QT intervals as possible. In particular, it is desirable that there be no significant QT/QTc interval prolongation. Therefore, new agents with improved safety and efficacy are needed for a more effective treatment of OAB.

Fesoterodine is known in the art for its potency in treating urinary incontinence. However, Fesoterodine may exhibit substantial degradation under stress conditions, e.g., in a humid environment and at increased temperature. It is believed that hydrolyzation and oxidation are among the major mechanisms resulting in degradation.

U.S. Patent Application publication No. 2011/0086130 discloses solid state form of a mandelate salt of fesoterodine.

International (PCT) Application No. WO 2010/010464 discloses a process for the preparation of pure fesoterodine fumarate which is substantially free of fesoterodine dehydroxy impurity. The application also discloses compositions containing said pure fesoterodine fumarate having a 90 volume percent of particles (D₉₀) with size less than or equal to about 200 microns.

International (PCT) Application No. WO 2011/050961 discloses a Pharmaceutical composition containing fesoterodine and/or its metabolites and fiber.

U.S. Pat. No. 7,807,715 discloses fesoterodine tablet formulations containing a stabilizer against hydrolysis, wherein the stabilizer is preferably a sugar alcohol, such as sorbitol and xylitol. Furthermore, the drug needs to be involved in artificial polymer, so that an extended release could be achieved in a matrix. However, it was found that the amount of decomposition products can only be controlled if the proposed formulations were prepared by classical wet granulation. A direct compression or dry granulation also resulted in an identical composition to higher amounts of undesirable decomposition products (compared to the wet granulation).

The prior art references emphasize on using stabilizers, for example—sugar alcohols (sorbitol or xylitol), polydextrose, isomalt and dextrose in order to prepare a stable composition of fesoterodine. There is no disclosure or teaching/suggestion, however, in the art about how to develop stable formulations of fesoterodine without employing stabilizers which can also exhibit drug release over an extended period of time. Also there is no disclosure or teaching/suggestion in the art about how to develop stable formulations of fesoterodine with specific particle size of fesoterodine of salts thereof.

Therefore, it would be desirable to develop new pharmaceutical compositions comprising Fesoterodine that are more stable against degradation over storage period and simultaneously, provide desired extended release of the drug.

### SUMMARY OF THE INVENTION

In one general aspect, there is provided particles of fesoterodine or salts thereof, wherein at least 90% of the total amount of the particles by volume (D₉₀) are having size greater than about 200 microns.

In another general aspect, there is provided particles of fesoterodine fumarate, wherein at least 90% of the total amount of the particles by volume (D₉₀) are having size greater than about 200 microns.

In another general aspect, there is provided particles of fesoterodine or salts thereof, wherein at least 90% of the total amount of the particles by volume (D₉₀) are having size greater than about 200 microns and less than about 500 microns;

In another general aspect, there is provided particles of fesoterodine or salts thereof wherein at least 90% of the total amount of the particles by volume (D₉₀) are having size in the range of about 300 microns and about 400 microns.

In another general aspect, there is provided an extended release pharmaceutical composition comprising particles of fesoterodine or salts thereof and one or more rate controlling polymers, wherein at least 90% of the total amount of particles of fesoterodine or salts thereof by volume (D₉₀) are having size greater than about 200 microns.

In another general aspect, there is provided an extended release pharmaceutical composition comprising particles of fesoterodine or salts thereof and one or more rate controlling polymers, wherein at least 90% of the total amount of particles of fesoterodine or salts thereof by volume (D₉₀) are having size greater than about 200 microns and less than about 500 microns.

In another general aspect, there is provided an extended release pharmaceutical composition comprising particles of fesoterodine or salts thereof and one or more rate controlling polymers, wherein at least 90% of the total amount of particles of fesoterodine or salts thereof by volume (D₉₀) are having size in the range of about 300 microns and about 400 microns.

In another general aspect, there is provided an extended release pharmaceutical composition comprising particles of fesoterodine or salts thereof and one or more rate controlling polymers, wherein at least 90% of the total amount of particles of fesoterodine or salts thereof by volume (D₉₀) are having size greater than about 200 microns and the composition is free of any stabilizer.

In another general aspect, there is provided an extended release pharmaceutical composition comprising particles of fesoterodine or salts thereof and one or more rate controlling polymers, wherein at least 90% of the total amount of particles of fesoterodine or salts thereof by volume (D₉₀) are having size greater than about 200 microns and the composition comprises about 10% to about 40% w/w of lactose by total weight of the composition.

In another general aspect, there is provided an extended release pharmaceutical composition comprising particles of fesoterodine or salts thereof and one or more rate controlling polymers, wherein at least 90% of the total amount of particles of fesoterodine or salts thereof by volume (D₉₀) are having size greater than about 200 microns and the composition comprises about 45% to about 70% w/w mixture of lactose and microcrystalline cellulose by total weight of the composition.

In another general aspect, there is provided an extended release pharmaceutical composition comprising about 1% to 3% w/w particles of fesoterodine or salts thereof, about 15% to 40% w/w of microcrystalline cellulose, about 10% to about 40% w/w of lactose, about 1% to 10% w/w of corn starch, about 25% to 60% w/w of hydroxypropyl methylcellulose, about 0.5% to about 5% w/w of povidone K 30, about 0.1% to about 2% w/w of talc, and about 0.5% to 3% w/w of magnesium stearate, wherein at least 90% of the total amount of particles of fesoterodine or salts thereof by volume (D₉₀) are having size greater than about 200 microns.

In another general aspect there is provided an extended release pharmaceutical composition comprising particles of fesoterodine or salts thereof and one or more rate controlling polymers, wherein at least 90% of the total amount of particles of fesoterodine or salts thereof by volume (D₉₀) are having size greater than about 200 microns and the composition exhibits no significant difference in rate and/or extent of absorption of fesoterodine as compared to extended release formulation commercially marketed under the trade name TOVIAZ®.

In another general aspect there is provided an extended release pharmaceutical composition comprising particles of fesoterodine or salts thereof and one or more rate controlling polymers, wherein at least 90% of the total amount of particles of fesoterodine or salts thereof by volume (D₉₀) are having size greater than about 200 microns and the composition retains at least 80% of the potency of fesoterodine in the pharmaceutical composition after storage for three months at 40° C. and 75% relative humidity or stressed stability conditions.

Embodiments of the present invention may include one or more of the following features. For example, the pharmaceutical composition may further include one or more pharmaceutical acceptable excipients. The pharmaceutical acceptable excipients may include diluents, disintegrants, plasticizers, binder, glidants, fillers and lubricants.

In another general aspect there is provided a process for the preparation of an extended release pharmaceutical composition of fesoterodine or salts thereof comprising—
(a) preparing granules comprising particles of fesoterodine or salts thereof and one or more rate controlling polymers; and
(b) formulating resulting granules into suitable dosage form,
   wherein at least 90% of the total amount of particles of fesoterodine or salts thereof by volume (D₉₀) are having size greater than about 200 microns.

In another general aspect there is provided a process for the preparation of an extended release pharmaceutical composition of fesoterodine or salts thereof comprising—
(a) preparing granules comprising particles of fesoterodine or salts thereof and one or more rate controlling polymers without using aqueous granulation solution; and
(b) formulating resulting granules into suitable dosage form,
   wherein at least 90% of the total amount of particles of fesoterodine or salts thereof by volume (D₉₀) are having size greater than about 200 microns.

In another general aspect there is provided a process for the preparation of an extended release pharmaceutical composition of fesoterodine or salts thereof comprising—
(a) preparing a blend comprising particles of fesoterodine or salts thereof and one or more rate controlling polymers; and
(b) formulating resulting blend into suitable dosage form,
   wherein at least 90% of the total amount of particles of fesoterodine or salts thereof by volume (D₉₀) are having size greater than about 200 microns.

Embodiments of the present invention may include one or more of the following features. For example, the pharmaceutical composition may further include one or more pharmaceutical acceptable excipients. The pharmaceutical acceptable excipients may include diluents, disintegrants, plasticizers, binder, glidants, fillers and lubricants.

In another general aspect of the invention there is provided a method of treating overactive bladder or a symptom thereof in a subject in need thereof, the method comprises administering an extended release pharmaceutical composition comprising particles of fesoterodine or salts thereof and one or more rate controlling polymers, wherein at least 90% of the total amount of particles of fesoterodine or salts thereof by volume (D₉₀) are having size greater than about 200 microns.

The details of one or more embodiments of the present invention are set forth in the description below. Other features, objects and advantages of the invention will be apparent from the description.

### DETAILED DESCRIPTION OF THE INVENTION

The inventors of the present invention have surprisingly found that fesoterodine particles of particular size are able to significantly slow down the degradation of pharmaceutical compositions of fesoterodine over the storage period or under stress conditions.

The inventors have developed an extended release pharmaceutical composition of fesoterodine having drug particles having 90 volume percent of particles (D₉₀) with size greater than about 200 microns. The resulting composition was found to remain stable over the storage period.

Particularly, fesoterodine or salts thereof is stable against degradation and may retain at least 80% of the potency of fesoterodine or salts thereof in the pharmaceutical composition or the composition is stable against the degradation of fesoterodine or salts thereof after storage for six months at 40° C. and 75% relative humidity.

Moreover, such composition is also bioequivalent with marketed formulation, TOVAZ®, i.e. it exhibits no significant difference in rate and/or extent of absorption of fesoterodine as compared to TOVIAZ®.

Embodiments of the present invention relates to particles of fesoterodine or salts thereof, wherein at least 90% of the total amount of the particles by volume (D₉₀) are having size greater than about 200 microns.

In an embodiment, at least 90% of the total amount of particles of fesoterodine or salts thereof by volume (D₉₀) are having size greater than about 200 microns.

In a further embodiment, at least 90% of the total amount of particles of fesoterodine or salts thereof by volume (D₉₀) are having size in the range of greater than about 200 microns and less than about 500 microns.

In a further embodiment, at least 90% of the total amount of particles of fesoterodine or salts thereof by volume (D₉₀) are having size in the range of about 300 microns and about 400 microns.

The extended release pharmaceutical composition of the invention comprises particles of fesoterodine or salts thereof and one or more rate controlling polymers, wherein at least 90% of the total amount of particles by volume (D₉₀) are having size greater than about 200 microns.

In an embodiment, the extended release pharmaceutical composition comprises particles of fesoterodine or salts thereof and one or more rate controlling polymers, wherein at least 90% of the total amount of particles of fesoterodine or salts thereof by volume (D₉₀) are having size in the range of about 300 microns and about 400 microns.

It was also surprisingly found that the extended release pharmaceutical composition, devoid of any stabilizer, though exhibits excellent storage stability by careful selection of excipients with their optimum concentrations.

In an embodiment, the extended release pharmaceutical composition is free of any stabilizer.

In a further preferred embodiment, the extended release pharmaceutical composition is free of sorbitol, xylitol, polydextrose, isomalt or dextrose.

In a further embodiment, the extended release pharmaceutical composition of fesoterodine comprises mixture of lactose and microcrystalline cellulose in amount ranging from 45% to about 70% w/w of the composition.

In a further embodiment, the extended release pharmaceutical composition of fesoterodine comprises lactose in amount ranging from about 10% to about 40% w/w of the composition.

The term ‘fesoterodine’ used throughout the specification refers to fesoterodine, its pharmaceutically acceptable salts, pharmaceutically acceptable solvates, pharmaceutically acceptable hydrates, pharmaceutically acceptable enantiomers, pharmaceutically acceptable derivatives, pharmaceutically acceptable polymorphs and pharmaceutically acceptable prodrugs thereof. Preferable salt is fesoterodine fumarate, and more preferably, fesoterodine hydrogen fumarate.

In an embodiment, the extended release pharmaceutical composition comprises fesoterodine or salts thereof, preferably fesoterodine hydrogen fumarate, or the free base, in an amount of about 0.5-28 mg, or about 0.5-20 mg, preferably about 1-16 mg, about 1-12 mg, more preferably about 1-8 mg, and even more preferably about 2, about 4 or about 8 mg per dosage unit (based on the content of fesoterodine or salts thereof, e.g., fesoterodine hydrogen fumarate), or free base.

The term “extended release” as used hereinbefore and throughout the description includes sustained release, controlled release, modified release and delayed release. The term extended release means release of the active agent at such a rate that blood (e. g., plasma) levels are maintained within a therapeutic range but below toxic levels for at least about 4 hours, preferably at least about 6 hours after administration at steady-state. The term “steady-state” means that a plasma level for a given active agent has been achieved and which is maintained with subsequent doses of the drug at a level which is at or above the minimum effective therapeutic level and is below the minimum toxic plasma level for a given active agent. With regard to dissolution profiles, the first and second dissolution profiles (e. g., in the stomach and in the intestines) should each be equal to or greater than the minimum dissolution required to provide substantially equivalent bioavailability to a capsule, tablet or liquid containing the at least one active ingredient in an immediate-release form.

Suitable “rate controlling polymers” may include one or more of hydrophilic and hydrophobic polymers or mixtures thereof.

Suitable hydrophilic polymers may include one or more of cellulosic polymers/copolymers or its derivatives including methyl cellulose, hydroxypropyl methylcellulose, hydroxyethyl cellulose, hydroxypropyl cellulose, hydroxyethyl methylcellulose, carboxymethylcellulose, sodium carboxymethylcellulose; polyacrylates, methyl acrylates, polyethylene oxides, polyethylene glycols, chitosan, gums, starch derivatives, polyurethanes, galactomannans, polysaccharides, polyalcohols, acrylic acid or acrylamide derivatives and the like. The preferred hydrophilic polymer is hydroxypropyl methylcellulose or any commercially available grade thereof such as Methocel.

Suitable hydrophobic polymers include one or more of ethyl cellulose, glycerol palmitostearate, beeswax, glycowax, carnaubawax, hydrogenated vegetable oil, glycerol monostearate, stearylalcohol, glyceryl behenate, polyanhydrides, methyl acrylates and the like.

The polymers used can also be eroding or non-eroding or combination of both. The polymers, which may be used for bioadhesion, are described below.

Natural polymers include but are not limited to proteins (e.g., hydrophilic proteins), such as pectin, zein, modified zein, casein, gelatin, gluten, serum albumin, or collagen, chitosan, oligosaccharides and polysaccharides such as cellulose, dextrans, tamarind seed polysaccharide, gellan, carrageenan, xanthan gum, gum Arabic; hyaluronic acid, polyhyaluronic acid, alginic acid, sodium alginate.

When the bioadhesive polymer is a synthetic polymer, the synthetic polymer is typically selected from but are not limited to polyamides, polycarbonates, polyalkylenes, polyalkylene glycols, polyalkylene oxides, polyalkylene terephthalates, polyvinyl alcohols, polyvinyl ethers, polyvinyl esters, polyvinyl halides, polyvinylpyrrolidone, polyglycolides, polysiloxanes, polyurethanes, polystyrene, polymers of acrylic and methacrylic esters, polylactides, poly(butyric acid), poly(valeric acid), poly(lactide-co-glycolide), polyanhydrides, polyorthoesters, poly(fumaric acid), poly(maleic acid), and blends and copolymers or mixtures thereof.

Preferably, suitable polymers for use in the present invention include, but are not limited to, methyl cellulose, ethyl cellulose, hydroxypropyl cellulose, hydroxypropylmethyl cellulose, hydroxybutylmethyl cellulose, cellulose acetate, cellulose propionate, cellulose acetate butyrate, cellulose acetate phthalate, carboxymethyl cellulose, cellulose triacetate, cellulose sulfate sodium salt, poly(methyl methacrylate), poly(ethyl methacrylate), poly(butyl methacrylate), poly(isobutyl methacrylate), poly(hexyl methacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate) polyethylene, polypropylene, poly(ethylene glycol), poly(ethylene oxide), poly (ethylene terephthalate), polyvinyl acetate), polyvinyl chloride, polystyrene, polyvinyl pyrrolidone, and polyvinylphenol. Polylactides, polyglycolides and copolymers thereof, poly(ethylene terephthalate), poly(butyric acid), poly(valeric acid), poly(lactide-co-caprolactone), poly[lactide-co-glycolide], polyanhydrides (e.g., poly(adipic anhydride)), polyorthoesters, blends and copolymers thereof. Another group of polymers suitable for use as bioadhesive polymers but not necessarily limited to polymers having a hydrophobic backbone with at least one hydrophobic group pendant from the backbone. Suitable hydrophobic groups are groups that are generally non-polar. The amount of rate controlling polymer in the composition of the present invention ranges from about 25% to 60% w/w of the composition.

In an embodiment, the extended release of fesoterodine or salts thereof can be achieved by providing a matrix and/or coated cores comprising fesoterodine or salts thereof and one or more rate controlling polymer/s.

In a further embodiment, the extended release pharmaceutical composition comprises core comprising matrix of fesoterodine or salts thereof with one or more rate controlling polymer/s, which core is further coated with one or more rate controlling polymer/s.

In an embodiment, the extended release pharmaceutical composition is not particularly limited as long as it is an oral preparation. For example, capsules can be packed with one or more tablets, granules or fine granules based on the matrix type or coated cores of fesoterodine according to the present invention. Further, hard capsules can be packed with multiple small-diameter mini-tablets.

The composition can also be given a film coating as necessary. It should be noted that the presence or absence of a hydrophilic film coating on the extended release preparation according to the present invention has very little effect on the dissolution profile of fesoterodine or salts thereof.

The extended release pharmaceutical composition of fesoterodine or salts thereof is preferably developed into dosage forms such as matrix-cores/tablets/granules/pellets, coated cores/tablets/granules/pellets or multiple unit particles which can be filled into capsules or compressed to form minitablets or tablets.

Alternatively, the extended release pharmaceutical composition of fesoterodine or salts thereof can be developed using various osmotic-controlled release oral systems (OROS) known in the art.

The extended release pharmaceutical composition of fesoterodine may be prepared by processes known to the person having ordinary skill in the art of pharmaceutical technology such as direct compression, dry granulation, slugging, hot melt granulation, hot melt extrusion, fluidized bed granulation, extrusion-spheronization, spray drying and solvent evaporation.

The inventors have further surprisingly found that when fesoterodine cores are prepared without using aqueous based granulation, in particular, preparing granules of fesoterodine by dry mixing, dry granulation, direct compression, slugging or by non-aqueous granulation using organic solvent (e.g. alcohol, methylene chloride), resulting formulations possess improved stability.

In an embodiment, the process for preparing the stable extended release pharmaceutical composition of fesoterodine or salts thereof in accordance with the present invention comprising the steps of:
(a) preparing a blend of fesoterodine or salts thereof comprising one or more rate controlling polymers, and
(b) formulating resulting blend into suitable dosage form.

In an embodiment, the process for preparing the stable extended release pharmaceutical composition of fesoterodine or salts thereof in accordance with the present invention comprising the steps of:
(a) preparing granules of fesoterodine or salts thereof comprising one or more rate controlling polymers, and
(b) formulating resulting granules into suitable dosage form.

In a further embodiment, the process for preparing the stable extended release pharmaceutical composition of fesoterodine or salts thereof in accordance with the present invention comprising the steps of:
(a) preparing granules of fesoterodine or salts thereof comprising one or more rate controlling polymers without using aqueous granulation solution, and
(b) formulating resulting granules into suitable dosage form.

In a still further embodiment, the process for preparing the stable extended release pharmaceutical composition of fesoterodine or salts thereof in accordance with the present invention comprising the steps of:
(a) preparing granules of fesoterodine or salts thereof comprising one or more rate controlling polymers by dry mixing, without using an aqueous granulation solution, and
(b) formulating resulting granules into suitable dosage form.

The pharmaceutically acceptable excipients may include one or more diluents/fillers, lubricants, disintegrants, glidants, colorants, sweeteners, plasticizers and the like.

Suitable fillers may include, but not limited to one or more of microcrystalline cellulose, starch, corn starch, dibasic calcium phosphate, tribasic calcium phosphate, calcium carbonate, dextrose, kaolin, magnesium carbonate, magnesium oxide; sugars such as lactose (lactose monohydrate or lactose anhydrous) or sucrose; sugar alcohols such as mannitol, sorbitol, erythritol and the like.

Suitable disintegrants may include, but not limited to one or more of croscarmellose sodium, sodium starch glycolate, pregelatinized starch, sodium carboxymethyl cellulose, cross-linked polyvinylpyrrolidone and the like. Preferred diluents for use in the present invention are lactose, microcrystalline cellulose and corn starch. The amount of diluent in the composition ranges from about 1% to about 50% w/w of composition.

Suitable binders may include one or more of hydroxyethyl cellulose, hydroxypropyl cellulose, carbomers, dextrin, ethyl cellulose, methylcellulose, shellac, zein, gelatin, polymethacrylates, polyvinyl pyrrolidone (e.g. Povidone K 30), pregelatinized starch, sodium alginate, gums, synthetic resins and the like. Preferred binder for use in the present invention is polyvinyl pyrrolidone. The amount of binder in the composition ranges from about 0.5% to about 5% w/w of composition.

Suitable lubricants and glidants may include one or more of talc, metallic stearates such as magnesium stearate, calcium stearate, zinc stearate; colloidal silicon dioxide, finely divided silicon dioxide, stearic acid, hydrogenated vegetable oil, glyceryl palmitostearate, glyceryl monostearate, glyceryl behenate, polyethylene glycols, powdered cellulose, starch, sodium stearyl fumarate, sodium benzoate, mineral oil, magnesium trisilicate, kaolin; and the like. Preferred lubricants for use in the present invention are talc and magnesium stearate. The amount of lubricant in the composition ranges from 0.1% to 3% w/w of composition.

Suitable plasticizers may include, but not limited to one or more of polyols such as glycerol, propylene glycol, polyethylene glycol (PEG), urea, or other known plasticizers such as triethyl citrate, dibutyl or dimethyl phthalate or water.

Present invention further provides a method of treating overactive bladder or a symptom thereof in a subject in need thereof such as urinary incontinence, urinary urge incontinence, imperative urinary urge, and increased urinary frequency, the method comprises administering an extended release pharmaceutical composition comprising fesoterodine or salts thereof as substantially described herein before.

The invention is further illustrated by the following examples which are provided to be exemplary of the invention and do not limit the scope of the invention. While the present invention has been described in terms of its specific embodiments, certain modifications and equivalents will be apparent to those skilled in the art and are intended to be included within the scope of the present invention.

### EXAMPLE 1

TABLE 1

| | | Composition |
|---|---|---|
| Sr. No. | Ingredient | (% w/w) |
| 1 | Fesoterodine fumarate | 2.24 |
| 2 | Microcrystalline cellulose | 25.21 |
| 3 | Corn starch | 4.20 |
| 4 | Lactose monohydrate | 19.75 |
| 5 | Hypromellose | 42.02 |
| 6 | Povidone K 30 | 2.24 |
| 7 | Talc | 0.98 |
| 8 | Magnesium Stearate | 1.40 |
| 9 | Opadry | 1.96 |
| 10 | Purified water | Q.S. |
| | Total | 100.00 |

(Particle size data of Fesoterodine fumarate: D₁₀-47.3μ; D₅₀-156μ; D90-293μ)
Procedure: Mixture of fesoterodine fumarate, microcrystalline cellulose, corn starch, lactose monohydrate, hypromellose, and povidone K 30 was lubricated with magnesium stearate and talc. The lubricated blend was then compressed into tablets. The tablets were finally coated with Opadry dispersion in purified water.

The stability study of this formulation was conducted at 40° C./75% RH over the period of 6 months.

The amount of the impurities measured in the formulation after the storage period indicates that the formulation of the invention is stable under stress conditions.

### STABILITY DATA OF EXAMPLE 1

Pack: HDPE Bottle with desiccant canister

| | | Time in Months | | | | |
|---|---|---|---|---|---|---|
| Test | Specification with limits | Initial | 1 | 2 | 3 | 6 |
| Related | Diol - NMT 1.5% | BQL | 0.05 | 0.08 | 0.11 | 0.17 |
| substance | Diester - NMT 0.62% | ND | BQL | 0.06 | 0.06 | 0.10 |
| | Oxidized - NMT 0.62% | BQL | 0.06 | 0.09 | 0.07 | 0.08 |
| | Unknown - NMT 0.25% | BQL | BQL | BQL | BQL | BQL |
| | Total - NMT 2.0% | 0.0 | 0.11 | 0.23 | 0.24 | 0.35 |

Pack: Blister Pack of 6's (ALU foil with desiccant)

| | | Time in Months | | | | |
|---|---|---|---|---|---|---|
| Test | Specification | Initial | 1 | 2 | 3 | 6 |
| Related | Diol - NMT 1.5% | BQL | 0.06 | 0.09 | 0.15 | 0.15 |
| substance | Diester - NMT 0.62% | ND | BQL | BQL | 0.06 | 0.10 |
| | Oxidized - NMT 0.62% | BQL | 0.07 | 0.08 | 0.06 | 0.08 |
| | Unknown - NMT 0.25% | BQL | BQL | BQL | BQL | 0.05 |
| | Total - NMT 2.0% | 0.0 | 0.13 | 0.17 | 0.27 | 0.38 |

BQL: below quantitation limit
NMT: Not More Than
   The amount of impurity refers to the percent by weight of fesoterodine or salts thereof.

### COMPARATIVE EXAMPLE 1

The formulation was prepared with the same excipients in the same quantity as used in Example 1. Only difference is in the particle size of fesoterodine fumarate used in that formulation. In this comparative example, particle size of fesoterodine fumarate is used such that D₁₀-2.67μ; D₅₀-34.48μ; D₉₀-125.3μ.

The accelerated stability study of this formulation was conducted at 50° C./80% RH over the period of 1 month.

The amount of the impurities measured in the formulation after the storage period indicates that the formulation is not stable under stress conditions.

### STABILITY DATA OF COMPARATIVE EXAMPLE 1

Pack: HDPE Bottle with desiccant canister

| | | Time | | |
|---|---|---|---|---|
| Test | Specification | Initial | 1 Week | 1 Month |
| Related | Diol - NMT 1.5% | 0.08 | 0.79 | 5.34 |
| substance | Diester - NMT 0.62% | BQL | 0.11 | 0.67 |
| | Oxidized - NMT 0.62% | 0.05 | 0.06 | 0.08 |
| | Unknown - NMT 0.25% | BQL | 0.04 | 0.62 |
| | Total - NMT 2.0% | 0.13 | 1.06 | 7.01 |

BQL: below quantitation limit
NMT: Not More Than
The amount of impurity refers to the percent by weight of fesoterodine or salts thereof.

The amount of impurity refers to the percent by weight of fesoterodine or salts thereof.

1. A stable extended release pharmaceutical composition comprising particles of fesoterodine or salts thereof, one or more rate controlling polymers and one or more pharmaceutically acceptable excipients wherein at least 90% of the total amount of particles of fesoterodine or salts thereof by volume (D₉₀) are having size greater than about 200 microns.

2. The stable pharmaceutical composition as claimed in claim 1, wherein at least 90% of the total amount of particles of festerodine or salts thereof by volume (D₉₀) are having size greater than about 200 microns and less than about 500 microns.

3. The stable pharmaceutical composition as claimed in claim 1, wherein the composition is free of any stabilizer.

4. The stable pharmaceutical composition as claimed in claim 3, wherein the stabilizer is selected from sugar alcohols (sorbitol or xylitol), polydextrose, isomalt and dextrose.

5. The stable pharmaceutical composition as claimed in claim 1, wherein the rate-controlling polymer is one or more of a hydrophilic Polymer, a hydrophobic polymer or a combination thereof.

6. The stable pharmaceutical composition as claimed in claim 5, wherein the hydrophilic polymer comprises one or more of cellulosic polymers/copolymers or its derivatives including methyl cellulose; hydroxypropyl methylcellulose, hydroxyethyl cellulose, hydroxypropyl cellulose; hydroxypropyl methylcellulose, carboxymethylcellulose, sodium carboxymethylcellulose polyacrylates, methyl acrylates, polyethylene oxides, polyethylene glycols, chitosan, gums, starch derivatives, polyurethanes, galactomannans, polysaccharides, polyalcohols.

7. The stable pharmaceutical composition as claimed in claim 1, wherein the pharmaceutically acceptable excipients are selected from binders, fillers, lubricants, disintegrants, glidants or combinations thereof.

8. The stable pharmaceutical composition as claimed in claim 1, wherein the composition comprises about 10% to about 40% w/w of lactose by total wieght of the compostion.

9. The stable pharmaceutical composition as claimed in claim 8, wherein the composition comprises about 45% to about 70% w/w mixture of lactose and microcrystalline cellulose by total weight of the composition.

10. The stable pharmaceutical composition as claimed in claim 1, wherein the composition exhibits no significant difference in rate and/or extent of absorption of fesoterodine as compared to extended release formulation commercially marketed under the trade name TOVIAZ®.

11. The stable pharmaceutical composition as claimed in claim 1, wherein the composition retains at least 80% of potency of festerodine or salts thereof when stored at 40° C./75% RH for 6 months.

12. The stable pharmaceutical composition as claimed in claim 1, wherein the composition retains at least 80% of potency of festerodine or salts thereof when stored at 50° C./80% RH for 1 month.

13. The stable pharmaceutical composition as claimed in claim 1, wherein the composition contains less than 1.5% of diol by weight of festerodine or salts thereof as an impurity when stored at 50° C./80% RH for 1 month.

14. The stable pharmaceutical composition as claimed in claim 1, wherein the composition contains less than 1.5% of diol by weight of festerodine or salts thereof as an impurity when stored at 40° C./75% RH for 6 months.

15. The stable pharmaceutical composition as claimed in claim 1, wherein the composition contains less than 2% of total impurities by weight of festerodine or salts thereof when stored at 50° C./80% RH for 1 month.

16. The stable pharmaceutical composition as claimed in claim 1, wherein the composition contains less than 2% of total impurities by weight of fesoterodine or salts thereof when stored at 40° C./75% RH for 6 months.

17. The stable pharmaceutical composition as claimed in claim 1, wherein the fesoterodine salt is fesoterodine fumarate.

18. The stable pharmaceutical composition as claimed in claim 1, wherein the extended release dosage form comprises multiple-unit particles comprising fesoterodine or salts thereof mixed or coated with one or more rate-controlling polymers.

19. A process for preparing a stable pharmaceutical composition comprising fesoterodine or salts thereof, the process comprising mixing and/or granulating fesoterodine or salts thereof with one or more rate-controlling polymers, optionally with one or more pharmaceutically acceptable excipients, wherein the composition is free of sugar alcohol; wherein at least 90% of the total amount of particles of fesoterodine or salts thereof by volume (D₉₀) are having size greater than about 200 microns.

20. A process for preparing the stable pharmaceutical composition as claimed in claim 19, the process comprising the steps of:
a) mixing fesoterodine or salts thereof, one or more rate controlling polymers and one or more pharmaceutically acceptable excipients; excipients;
b) granulating the mixture to form granules; and
c) converting the granules prepared in step (b) into a suitable dosage form.

21. A process for preparing the stable pharmaceutical composition as claimed in claim 19, the process comprising the steps of:
(a) preparing a blend comprising particles of fesoterodine or salts thereof and one or more rate controlling polymers; and
(b) formulating the resulting blend of step (a) into a suitable dosage form.

22. A stable extended release pharmaceutical composition comprising:
about 1% to 3% w/w particles of festerodine or salts thereof;
about 15% to 40% w/w of microcrystalline cellulose;
about 10% to about 40% w/w of lactose;
about 1% to 10% w/w of corn starch;
about 25% to 60% w/w of hydroxypropyl methylcellulose;
about 0.5% to about 5% w/w of povidone K 30;
about 0.1% to about 2% w/w of talc; and
about 0.5% to 3% w/w of magnesium stearate,
wherein at least 90% of the total amount of particles of fesoterodine or salts thereof by volume (D₉₀)) are having size greater than about 200 microns and the composition is free of sugar alcohol.

23. A method of treating a patient suffering from overactive bladder by administering a therapeutically effective amount of a stable pharmaceutical composition as claimed in claim 1.
